(19) United States
(12) Patent Application Publication
(10) Pub. No.: US 2013/0059035 A1
(43) Pub. Date: Mar. 7, 2013

(54) CAPSAICIN COMPOSITION AND METHOD RELATED THERETO

(71) Applicant: Massoud Arvanaghi, Franklin Lakes, NJ (US)

(72) Inventor: Massoud Arvanaghi, Franklin Lakes, NJ (US)

(21) Appl. No.: 13/606,210

(22) Filed: Sep. 7, 2012

Related U.S. Application Data
(60) Provisional application No. 61/531,733, filed on Sep. 7, 2011.

(51) Int. Cl. A23L 1/307; C07K 14/00

(52) U.S. Cl. 426/52; 530/395; 426/648

(57) ABSTRACT

A weight reduction composition is provided including capsaicin bound by a glycoprotein matrix, green tea leaves, ginger powder, garlic powder, and turmeric powder. By binding capsaicin to a glycoprotein matrix, the efficacy of capsaicin to promote weight loss is achieved at a lower concentration than otherwise required through the use of untreated capsaicin. Accordingly, the benefits of a higher concentration of capsaicin can be attained without the unpleasant side effects.

### CROSS REFERENCE TO RELATED APPLICATION(S)

This application claims the benefit of U.S. Provisional Application No. 61/531,733, filed Sep. 7, 2011.

### FIELD OF THE INVENTION

The present invention relates in general to a composition to facilitate weight loss and in particular to a composition comprising capsaicin bound by a glycoprotein matrix and a method of making the same.

### BACKGROUND OF THE INVENTION

Glycosylated proteins are present in the extracellular matrices and cellular surfaces of many cells. Glycoproteins are organic compounds composed of both protein and carbohydrate attached together by a covalent bond. The oligosaccharide moieties of the glycoprotein are implicated in a wide range of cell to cell and cell to matrix recognition events. The present invention relates to capsaicin composition that can be used to deliver capsaicin to a host and to methods of making such composition.

Our metabolism is the speed by which the body converts or burns calories into energy to maintain life functions. Muscle and diet account for how quickly our metabolism processes. The more muscle, the higher the metabolism, because muscles require more energy to maintain themselves. But since lifting weights is not for everyone, dietary changes can help increase metabolism.

Metabolism has been long regarded as an iconic key to long-term weight loss. If one has a fast metabolism, such person is a privileged member of society who can eat tempting treats and barely gain fat. A fast metabolism is the secret behind the skinny bodies of the younger generations. But as we age, our metabolism slows, and for some, considerably.

Chili peppers are known for their poignant, spicy flavor that adds zest to dishes. For many years, various nutritionists, dieticians and weight loss experts claimed that spicy foods help burn fat. New research coming to light indicates that they may have been right as an active ingredient in chili peppers called capsaicin is found to aid in weight loss in terms of curbing appetite, revving up metabolism, and boosting fat loss. It is known that capsaicin strengthens lung tissues; helps relieve pain, and aid digestion and cleansing among other attributes.

Recent research in the International Journal of Obesity by Maastricht University in the Netherlands shows capsaicin can also decrease appetite (Sensory and Gastrointestinal Satiety Effects of Capsaicin on Food Intake. Westerterp-Plantenga M S, Smeets A, Lejune M P. Int J. Obes. (London). 2005 (6):682-8). These outcomes indicate capsaicin increases fat burning and weight loss. Capsaicin, a natural substance sourced from chili peppers, is said to support weight loss. This compound may help speed up metabolism and reduce fat tissue, as well as curb overeating by taming appetite.

In tests on rats fed a high-fat diet, the authors of a 2010 study from the Journal of Proteome Research found that capsaicin stimulated certain proteins known to break down fat and inhibited the actions of proteins involved in producing fat (Proteomic Analysis for Antiobesity Potential of Capsaicin on White Adipose Tissue in Rats Fed with a High Fat Diet. Jeong In Joo, Dong Hyun Kim, Jung-Won Choi and Jong Won Yun; J. Proteome Res., 2010, 9 (6), pp 2977-87).

Furthermore, a 2003 study found that capsaicin may help decrease ghrelin (a hormone involved in promoting hunger). These results indicate that capsaicin treatment caused sustained fat oxidation during weight maintenance compared with a placebo (Effect of capsaicin on Sustrate Oxidation and Weight Maintenance After Modest Body-Weight Loss in Human Subjects. Lejeune M P, Kovacs E M, Westerterp-Plantenga M S: Br. J. Nutri., 2003, 90(3):651-59). However, capsaicin treatment had no limiting effect on 3-month weight regain after modest weight loss. Another study conducted in 2009 mentioned in the Journal of Clinical Nutrition suggests that capsaicin reduces appetite, as a result, subjects ate less calories and fat loss ensued (Effects of Novel Capsinoid Treatment on Fatness and Energy Metabolism in Human: Possible Pharmacogenetic Implications. S. Snitker; et al,; Cmerican Clinical Journal of Nutrition, 2009).

Another study indicated that combination of red pepper and caffeine will aid losing weight (Combined Effects of Red Pepper and Caffeine Consumption on 24 h Energy Balance in Subjects Given Free Access to Foods. Yoshioka M, Doucet E, Drapeau V, Dionne I, Tremblay A. Br. J. Nutr. 2001, 85(2):203-11). Our metabolic rate is influenced by the food we consume. Food, in order to be processed and stored, requires energy to be expended. This increment in energy expenditure above the resting metabolic rate—released from the body as heat—is called thermic effect of food.

Obesity is the result of accumulation of extra and unused calories in the body in the form of fat. It depends on two processes-lipogenesis and adipogenesis. The conversion of unused calories into fat is known as lipogenesis, which takes place in adipocytes or fat cells. The fat storage capacity of a person increases with the rise in the number of adipocytes. The process of creation of adipocytes is known as adipogenesis.

In 2007, two Taiwanese food scientists provided evidence that capsaicin can prevent the formation of adipocytes, decreasing the amount of fat tissue and blood-fat levels, and inhibiting lipogenesis, which limits the fat storing capacity of an individual, thus preventing obesity.

Although capsaicin has been shown to aid weight loss, however, in many cases keeping the lost fat permanent has not been achieved. Capsaicin is a very pungent ingredient that may have many side effects. Furthermore, a large quantity of capsaicin is needed to control fat reduction. Hence, there is need to produce an already metabolized capsaicin that can exhibit the same or better characteristics of weight loss without its side effects and large dose. In addition, in order to increase the potency of capsaicin other ingredients such as caffeine are mixed with capsaicin to boost its activity. However, caffeine has its own drawbacks and side effects.

The present invention succeeds in providing a uniquely predigested capsaicin product without adding any other harmful additives such as caffeine. In the present invention, Capsaicin may be grown in a culture of Saccharomyces Cerevisiae.

### SUMMARY OF THE INVENTION

According to the present invention, the foregoing and other objects and advantages are obtained by utilizing a composition comprising capsaicin bound by a glycoprotein matrix, green tea leaves, ginger powder, garlic powder and turmeric powder.

According to another aspect of the invention, a method is provided for preparing a glycoprotein-matrix bound composition, comprising the steps of preparing a nutrient media comprising green tea leaves, ginger powder, garlic powder, turmeric powder, and water. The nutrient media is heated for thirty minutes at a temperature between 145° and 150° F. Oleoresin capsaicin is added to the nutrient media. The nutrient media is stirred at approximately 140° F. for 1 hour. The nutrient media is removed from a heat source and permitted to cool to a temperature between 90° and 95° F. Citrus concentrate is added to the nutrient media. A yeast solution is formed by adding an active Saccharomyces Cerevisiae yeast to water with continuous aeration to form an aqueous solution. Next, maltodextrin is added to the yeast solution. The nutrient media solution is inoculated into the active yeast solution to form a live culture solution. The mixture is permitted to bloom and metabolize for approximately four and one half hours at a temperature of between 90° and 95° F. GFT chromium yeast is added to the culture solution, permitting the live culture to grow for an extra hour at a temperature of between 90° and 95° F. Proteolytic enzyme is then added to the culture solution, permitting the live culture to grow for 30 minutes at a temperature of between 90° and 95° F. with constant stirring. The active growth is stopped by heating the culture solution to a temperature between 145° and 155° F. for two hours with continuous stirring. Next, the culture solution is homogenized in a shearing pump for 30-60 minutes. The resulting product is spray dried for 3 hours.

### BRIEF DESCRIPTION OF THE DRAWINGS

The invention will become more readily apparent from the following description of preferred embodiments thereof shown, by way of example only, in the accompanying drawings wherein:

FIG. 1 is a theoretical chemical reaction according to the instant invention.

### DETAILED DESCRIPTION OF THE INVENTION

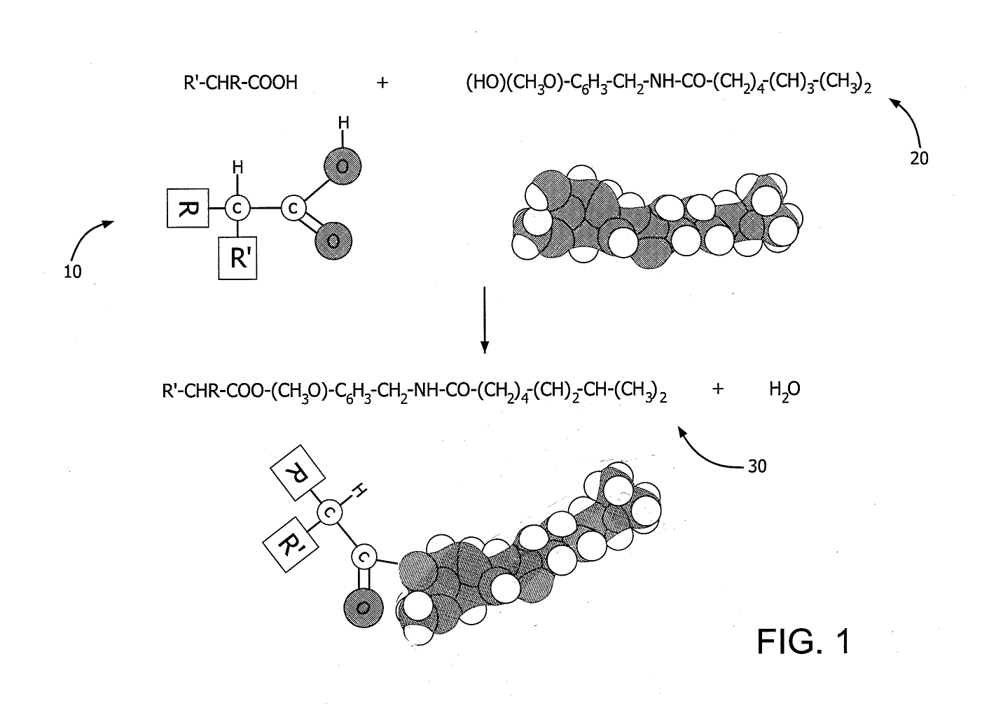

In accordance with the present invention, a capsaicin composition is provided. The composition includes a glycoprotein matrix bound to the capsaicin. Not intending to be bound by the theory, it is believed that the glycoprotein matrix bound to capsaicin permits greater bioavailability and absorption of the compound in the human body when compared to the administration of the same dosage of untreated capsaicin. The use of a glycoprotein matrix for increasing the bioactivity and stability of a compound is known in the art, as evidenced by the summary and detailed description disclosed in U.S. Pat. No. 6,942,856. However, the use of a glycoprotein matrix with capsaicin is not taught in the prior art and permits lower dosages of the instant composition to be administered to humans, while maintaining the same beneficial effect of a higher dosage of untreated capsaicin, but without the unwanted side effects resulting from ingesting larger amounts of capsaicin in the untreated form. The proposed theoretical schematic of the reaction between capsaicin and a yeast glycoprotein according to the instant invention is depicted in FIG. 1. A yeast glycoprotein, generally identified by reference numeral 10 is depicted with a protein side chain “R” and a protein chain “R′”. When combined with a capsaicin molecule, generally identified by reference numeral 20, the hypothetical resultant molecule is depicted as reference numeral 30. Water is also believed to be produced (not depicted).

The glycoprotein matrix can be produced by microorganisms, such as yeast or bacteria. The preferred microorganism is Saccharomyces Cerevisiae. The microorganism can, in other embodiments be selected from the genus Saccharomycecetacease, preferably Saccharomyces cerevisiae, Saccharomyces boulardii sequela and/or Saccharomyces torula. The composition of the invention can also include stabilizers or additives to improve its properties. For example, in a preferred embodiment, the composition of the invention also includes green tea, ginger, GTF Chromium yeast, garlic, turmeric, and citrus extract. Without being bound by the theory, it is believed that the combination of these stabilizers or additives increase the bioavailability, absorption and retention of these components, while at the same time making the capsaicin disclosed herein more effective than ingesting any of the components individually in either a form treated by the method of this invention or in an untreated form.

A method is also provided for preparing a capsaicin-containing composition. The method includes binding at least one capsaicin to a glycoprotein matrix. In a preferred embodiment, the glycoprotein matrix is formed by glycoprotein-producing microorganism. In addition to Saccharomyces Cerevisiae, many other single-celled plants, including Lactobacillus reuteri, Lactobacillus ferintoshensis, Lactobacillus bulgaricus and combinations thereof can be used in another embodiment. Thus, the binding occurs by combining the capsaicin with a glycoprotein producing microorganism under conditions such that the microorganism will produce glycoprotein.

Organoliptic Characteristics:

A typical analytical composition of the invention is as follows:

Total Capsaicinoids: 20,000-25,000 Scoville heat units (“SHU”). An SHU is a measurement of the spicy heat (or piquance) of a chili pepper. The number of SHUs indicate the amount of capsaicin present. Capsaicin is a chemical compound that stimulates chemoreceptor nerve endings in the skin, especially the mucus membranes; pure capsaicin has a SHU rating of 16,000,000 SHU. The composition is composed of typically;

Nordihydrocapsacin: 9,000-11,000 SHU

Capsaicin: 500-2,500 SHU

Dihydrocapsaicin: 10,000-12,000 SHU

Total Capsaicin content: 0.4-0.7% (by weight) (though, in other embodiments, between 0.05 and 5% are effective)

Protein Content: 5-25% (by weight)

Carbohydrate Content: 40-60% (by weight)

Fiber Content: 12-18% (by weight)

Fat Content: 0.5-5% (by weight)

Moisture Content: 5-12% (by weight)

Ash Content: 2-6% (by weight)

Mineral Content:

Calcium: 500-1000 ppm
Chromium: 250-400 ppm
Potassium: 9,000-13,000 ppm
Heavy Metals (as lead): <5 ppm
Arsenic: <10 ppm

### EXAMPLE 1

This example demonstrates the preparation of a composition of the invention in the preferred embodiment. The particular method employs 3.5 kilograms of dry ingredients to yield approximately 2.9 kilograms of capsaicin-containing composition.

A nutrient media-containing capsaicin was first prepared. 150 g green tea leaves(though, in other embodiments between 10 and 250 g can be utilized), 200 g ginger powder (though, in other embodiments between 0 and 500 g can be utilized), 100 g garlic powder (though, in other embodiments between 0 and 500 g can be utilized), and 100 g turmeric powder (though, in other embodiments between 10 and 500 g can be utilized) were added slowly to 5 liters of water heated to 145-150° F. (though, in other embodiments, the temperature can range from 100-150° F.). In other embodiments, it is understood that an individual having ordinary skill in the art could substitute powdered products for correlated amounts of extracts or concentrates. After 15 to 120 minutes (where 30 minutes are preferred), 120 g (3.43%) oleoresin capsaicin (250,000 SHU) was added to the solution. In other embodiments of the invention, between 10-500 g of oleoresin capsaicin may be added at 250,000 SHU. The solution was stirred at about 140° F. for preferably 1 hour (though between 45 and 180 minutes are effective in other embodiments) and allowed to cool to 90-95° F. (in other embodiments, the cooled temperature can range from 80-100° F.). In the preferred embodiment, 500 g citrus concentrate was then added, but this may range from 250 to 1000 g.

An active yeast solution was then prepared. 1250 g active live baker's yeast, Saccharomyces Cerevisiae, was added to 8 liters H2O with continuous aeration to form an aqueous solution. In another embodiment Lactobacillus can be used as a substitute for live baker's yeast. 800 g maltodextrin was then added. (In other embodiments, between 800 and 1200 g can be added.) In other embodiments, molasses or cane sugar can be used as effective carbohydrates to substitute for maltodextrin.

The nutrient media-containing capsaicin was then inoculated at a very slow rate into the active yeast solution to form a live culture solution. The mix was allowed to bloom and metabolize for 4.5 hours at 90-95° F. In other embodiments, the mix may be allowed to bloom and metabolize for between 3 and 6 hours. 250 g of GTF chromium yeast was added (though, in other embodiments, between 10 and 750 g can be used) and the mixture was allowed to grow for an extra one hour at 90-95° F. In other embodiments, this time can range from 30 to 180 minutes). 30 g proteolytic enzyme (Fungal Protease) was then added (though, in other embodiments, between 20 and 75 g can be used) and allowed to react for 30 minutes at 90-95° F. with constant stirring. In other embodiments, the reaction can occur for 10 to 90 minutes. Active growth was then stopped by heating the solution to 145-155° F. for 2 hours with continuous stirring. In other embodiments, the active growth can be stopped by heating the solution for between 1 and 3 hours.

The solution was then homogenized in a shearing pump for approximately 30-60 minutes (though this may range from 10 to 180 minutes in other embodiments) and spray dried for 3 hours (where the spray drying may range from 2 to 4 hours in other embodiments). In another embodiment, freeze drying can be used as an alternative to spray drying. The resulting product was a fine light brown to tan powder, which was analyzed for capsaicin content as described above.

### EXAMPLE 2

This example demonstrates the preparation of a composition of the invention. The particular method employs 3.5 kilograms of dry ingredients to yield approximately 2.95 kilograms of capsaicin-containing composition.

A nutrient media-containing capsaicin was first prepared. 180 g green tea, 190 g ginger powder, 180 g garlic powder, and 400 g turmeric powder were added slowly to 5 liters H2O heated to 100-105° F. After 60 minutes, 40 g (1.143%) oleoresin capsaicin (250,000 units) was added to the solution. The solution was stirred at about 105° F. for approximately 1 hour and allowed to cool to 90-95° F. 600 g citrus concentrate was then added.

An active culture solution was then prepared. 1000 g active live yogurt culture, Lactobacillus Bulgaricus, was added to 8 liters H2O to form an aqueous solution. 900 g maltodextrin was then added.

The nutrient media-containing capsaicin was then inoculated at a very slow rate into the active solution to form a live culture solution. The mix was allowed to metabolize for 4 hours at 85-95° F. 10 g of GTF chromium yeast was added and the mixture was allowed to grow for an extra 60 minutes at 85-95° F. Active growth was then stopped by heating the solution to 100-105° F. for one hour with continuous stirring.

The solution was then homogenized in a shearing pump for approximately 30-60 minutes and spray dried for approximately 2.5 hours. The resulting product was a fine light tan powder, which was analyzed for capsaicin content.

### EXAMPLE 3

This example demonstrates the preparation of a composition of the invention. The particular method employs 3.5 kilograms of dry ingredients to yield approximately 3.2 kilograms of capsaicin-containing composition.

A nutrient media-containing capsaicin was first prepared. 225 g green tea, 420 g ginger powder, 100 g garlic powder, and 60 g turmeric powder were added slowly to 4.5 liters H2O heated to 105-120° F. After 45 minutes, 25 g (0.7143%) oleoresin capsaicin (250,000 units) was added to the solution. The solution was stirred at about 100° F. for approximately 1.5 hour and allowed to cool to 90-95° F. 250 g citrus concentrate was then added.

An active yeast solution was then prepared. 1250 g active live baker's yeast, Saccharomyces Cerevisiae, was added to 8 liters H2O to form an aqueous solution. 800 g maltodextrin was then added.

The nutrient media-containing capsaicin was then inoculated at a very slow rate into the active yeast solution to form a live culture solution. The mix was allowed to bloom and metabolize for 5 hours at 85-90° F. 325 g of GTF chromium yeast was added and the mixture was allowed to grow for an extra 30 minutes at 85-90° F. 45 g proteolytic enzyme (Fungal Protease) was then added and allowed to react for 25 minutes at 85-90° F. with constant stirring. Active growth was then stopped by heating the solution to 105-115° F. for 2 hours with continuous stirring.

The solution was then homogenized in a shearing pump for approximately 15-45 minutes and spray dried for approximately 3 hours. The resulting product was a fine light brown to tan powder, which was analyzed for capsaicin content.

The compositions of this invention are effective in achieving weight loss in human subjects without the side effects of larger dosages of capsaicin and other compounds that would be necessary to achieve the same effect in an untreated form.

What is claimed is:

1. A composition for facilitating weight loss comprising:
capsaicin bound by a glycoprotein matrix.

2. The composition of claim 1 further comprising:
green tea leaves;
ginger powder;
garlic powder;
turmeric powder;
water.

3. The composition of claim 2 where the weight of green tea leaves ranges from 10 to 250 g.

4. The composition of claim 2 where the weight of ginger powder ranges from 0 and 500 g.

5. The composition of claim 2 where the weight of garlic powder ranges from 0 to 500 g.

6. The composition of claim 2 where the weight of turmeric powder ranges from 10 to 500 g.

7. A method for preparing a glycoprotein-matrix bound composition for facilitating weight loss, wherein said method comprises the steps of:
preparing a nutrient media comprising:
green tea leaves;
ginger powder;
garlic powder;
turmeric powder; and
water
heating the said nutrient media for thirty minutes at a temperature between 100° and 150° F.;
adding oleoresin capsaicin to the nutrient media;
stirring the nutrient media for 1 hour;
cooling the nutrient media to a temperature between 80° and 100° F.;
adding citrus concentrate to the nutrient media;
forming an active solution by adding a glycoprotein producer to water with continuous aeration to form an aqueous solution;
adding a carbohydrate to the said active solution;
inoculating the nutrient media solution into the active solution to form a culture solution;
permitting the mixture to bloom and metabolize for approximately three to six hours at a temperature of between 90° and 95° F.;
adding GFT chromium yeast to the culture solution and permitting the live culture to grow for 30 to 180 minutes at a temperature of between 90° and 95° F.;
adding proteolytic enzyme to the culture solution and permitting the live culture to grow for 10 to 90 minutes at temperature of between 90° and 95° F. with continuous stirring;
stopping active growth in the culture solution by heating the culture solution to a temperature between 145° and 155° F. for between one and three hours with continuous stirring;
homogenizing the culture solution for between 10 and 180 minutes; and
drying the culture solution.

8. The nutrient media of claim 7 wherein the concentration of green tea leaves is between 2 and 50 grams per liter.

9. The nutrient media of claim 7 wherein the carbohydrate is selected from the group consisting of maltodextrin, molasses, and cane sugar.

10. The nutrient media of claim 9 wherein the concentration of the carbohydrate ranges from 100 to 150 grams per liter.

11. The nutrient media of claim 7 wherein the concentration of ginger powder is between 0 and 100 grams per liter.

12. The nutrient media of claim 7 wherein the concentration of garlic powder is between 0 and 100 grams per liter.

13. The nutrient media of claim 7 wherein the concentration of turmeric powder is between 2 and 100 grams per liter.

14. The nutrient media of claim 7 wherein the concentration of oleoresin capsaicin is between 5 and 100 grams per liter.

15. The nutrient media of claim 7 wherein the concentration of citrus concentrate is 100 grams per liter.

16. The solution of claim 7 wherein the glycoprotein producer is selected from the group consisting of active Saccharomyces Cerevisiae yeast, Saccharomyces boulardii sequel, Saccharomyces torula, Lactobacillus reuteri, Lactobacillus ferintoshensis, and Lactobacillus bulgaricus.

17. The solution of claim 16 wherein the concentration of Saccharomyces Cerevisiae is 218.75 grams per liter.

18. The culture solution of claim 7 wherein the concentration of GTF chromium yeast is between 0.77 and 57.70 grams per liter.

19. The culture solution of claim 7 wherein the concentration of proteolytic enzyme is between 1.54 and 5.77 grams per liter.

20. The culture solution of claim 7 wherein the drying occurs by spray drying the resulting product for between two and four hours.

21. The culture solution of claim 7 wherein the drying occurs by freeze drying the resulting product.
